(19) United States
(12) Patent Application Publication
(10) Pub. No.: US 2007/0173717 A1
(43) Pub. Date: Jul. 26, 2007

(54) Medical apparatus with a multi-modality interface

(75) Inventors: Estelle Camus, Erlangen (DE); Oliver Meissner, Munchen (DE); Martin Ostermeier, Buckenhof (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/655,958

(22) Filed: Jan. 19, 2007

(30) Foreign Application Priority Data
Jan. 23, 2006 (DE) 10 2006 003 180.6

(51) Int. Cl. A61B 5/05

(52) U.S. Cl. 600/427

(57) ABSTRACT

The invention relates to a medical examination and/or treatment apparatus comprising an x-ray image recording device, a radiation source, a radiation receiver, a control and processing device and an image generating device, wherein at least one invasive sensor device with an imaging system can be connected to the medical examination and/or treatment apparatus via a multi-modality interface which is implemented as a universal interface via which different invasive sensor devices can be connected.

### CROSS REFERENCE TO RELATED APPLICATIONS

This application claims priority of German application No. 10 2006 003 180.6 filed Jan. 23, 2006, which is incorporated by reference herein in its entirety.

### FIELD OF THE INVENTION

The invention relates to a medical examination and/or treatment apparatus comprising an x-ray image recording device, a radiation source, a radiation receiver, a control and processing device and an image generating device.

### BACKGROUND OF THE INVENTION

In the context of interventional cardiology and radiology, various invasive intravascular imaging methods such as OCT, IVUS and various diagnostic methods such as pressure wire are available. These imaging and diagnostic methods can be used alone or in conjunction with a therapeutic procedure.

The various methods mentioned can be used depending on the physician's lines of enquiry. It is therefore desirable that the different invasive imaging methods are available to the physician. Efforts are made to integrate these various invasive imaging methods and the x-ray image recording device into a workflow.

DE 102 55 957 A1 describes a medical examination and/or treatment system in which an x-ray image recording device and an image recording system for optical coherence tomography (OCT) are integrated, so that a common image representation is possible.

In the brochure “AXIOM Artis Angiography Systems, Integrated Ultrasound Functionality for Enhanced Clinical Workflow” (Siemens AG, Erlangen, 2003) an integrated system comprising an x-ray image recording device and a non-invasive ultrasound system is described. In this system the ultrasound heads including the electronic unit for sending and receiving the ultrasound signals as well as a joystick for operating the ultrasound equipment are on the patient table. The ultrasound equipment control device is connected via long cables installed in the patient table and the floor and is located in a separate control room from which the ultrasound equipment can also be operated. A monitor in the monitor array in the examination room is connected to the control computer. The control device is connected to the hospital network in order to be able to transmit the examination images over the network.

The conventional integrated systems described, each having an x-ray image recording device and an invasive or non-invasive catheter, are only individual solutions. Invasive sensor devices are generally implemented as independent units which are moved into the examination room when required. However, this has a number of disadvantages. The various non-compatible units increase the amount of space required. The unit is normally some distance away from the patient table during the examination or therapy, which means that the physician has to turn round to operate it or view images. As in most cases no network connection is present, it is not easily possible to exchange examination images or other results. The different equipment-specific and proprietary user interfaces make the equipment difficult to use, as the physical has to adjust to each new device. All in all, as integrated evaluation or visualization is impossible, the workflow is non-optimal.

### SUMMARY OF THE INVENTION

The object of the invention is to create a medical examination and/or treatment apparatus which avoids the disadvantages mentioned and enables a better workflow.

This object is inventively achieved for a medical examination and/or treatment apparatus of the type described in the introduction by providing for at least one invasive sensor device with an image recording system to be connected to it via a multi-modality interface implemented as a universal interface via which different invasive sensor devices can be connected.

The invention is based on the knowledge that the incompatibilities that exist in the prior art can be eliminated by creating a universal, standardized interface which is implemented in the medical examination and/or treatment apparatus and to which a plurality of different invasive sensor devices can be connected. The invention enables a number of different invasive sensor devices to be easily connected to the examination and/or treatment apparatus when required and used alternately or consecutively.

According to the invention such an invasive sensor device can be implemented as a catheter, in particular a flexible catheter. On the other hand, such an invasive sensor device can also be implemented as a fixed device, in particular as a needle.

In order to ensure that the individual invasive sensor devices are easily interchangeable, it can be provided that the multi-modality interface comprises a plug-in connection. The individual sensor devices or catheters can each have a plug which can be inserted in the corresponding jack on the multi-modality interface.

Particularly flexible deployment of the examination and/or treatment apparatus according to the invention is provided if a sensor device from the following group can be connected: IVUS, OCT, OFDI, ICE, pressure wire, angioscopy, NIRF imaging, intravascular MR. These sensor devices which are used for quite different intravascular imaging methods or diagnostic procedures can be flexibly connected to the medical examination and/or treatment apparatus and disconnected again and interchanged.

In order to improve handling and operating of the examination and/or treatment apparatus, it can be provided that it incorporates a user interface and/or an interface to an evaluating computer and/or an interface to a tracking system and/or to a magnetic navigation device and/or to an ECG recorder. This means that virtually the entire workflow can be controlled and processed via the examination and/or treatment apparatus. The user can perform all the inputs at a single user interface, thereby obviating the need to constantly re-acquaint himself with different equipment-specific or proprietary user interfaces. With the medical examination and/or treatment apparatus according to the invention, a user interface can comprise a touch-sensitive screen and/or a joystick.

### BRIEF DESCRIPTION OF THE DRAWINGS

Further advantages and details of the invention will now be explained with reference to the accompanying schematic drawings in which:

FIG. 1 shows a first embodiment of a medical examination and/or treatment apparatus according to the invention, and

FIG. 2 shows a second embodiment of a medical examination and/or treatment apparatus

### DETAILED DESCRIPTION OF THE INVENTION

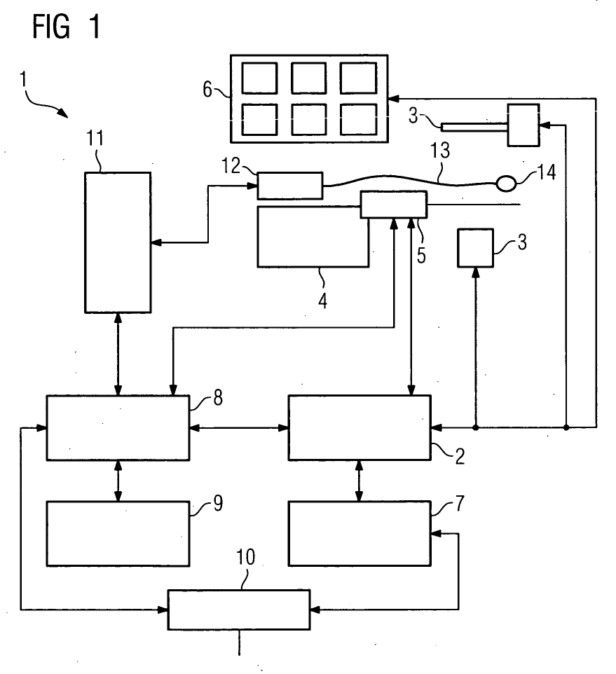

The medical examination and/or treatment apparatus 1 schematically illustrated in FIG. 1 comprises an x-ray image recording device 2, a radiation source and a radiation receiver which are incorporated in a C-arm 3, and a patient positioning table 4.

The x-ray image recording device 2, which includes a control and processing device, is connected in known manner to a user interface 5 disposed in proximity to a patient table 4, said user interface 5 having operator buttons, a touch-sensitive monitor and a joystick as input devices. The x-ray image recording device 2 is linked to a monitor array 6 on which a plurality of examination images can be displayed simultaneously.

The x-ray image recording device 2 is also connected to an ECG machine 7. The x-ray image recording device 2 is adjacently connected to a multi-modality interface 8 to which different catheters can be connected. The multi-modality interface 8 is linked to an evaluating computer 9 implemented as an image generating device. Image processing is performed by 3D reconstruction software, and x-ray recordings can likewise be merged with catheter system Images.

The multi-modality interface 8 is connected to other computers via a schematically illustrated network 10, similarly the x-ray image recording device 2 is connected to the network 10 via the ECG machine 7.

The multi-modality interface 8 is implemented as a standardized interface with a plug-in connection and enables different invasive sensor devices 11 implemented as catheters in the embodiment shown to be connected. These are catheters for different modalities such as IVUS (intravascular ultrasound), OCT (optical coherence tomography), OFDI (optical frequency domain imaging), ICE (intracoronary echography), etc. Connected to the sensor device 11 is a patient interface unit 12 which withdraws the relevant catheter again. Such a catheter 13 is schematically shown in FIG. 1 with magnetic coils 14 at its end so that the current position of the catheter 13 can be detected via a tracking system.

In other variants the multi-modality interface 8 implemented as a standardized interface can also be expanded to include further standardized or modality-specific interfaces. As shown in FIG. 1, the multi-modality interface 8 as well as the x-ray image recording device 2 is directly connected to the user interface 5.

The user interface 5 has a touch-sensitive screen and a joystick. The connection between the multi-modality interface 8 and the user interface 5 can be established directly or via the x-ray image recording device 2. The user interface 5 can be disposed either on the patient table 4 or additionally in a control room at a distance therefrom.

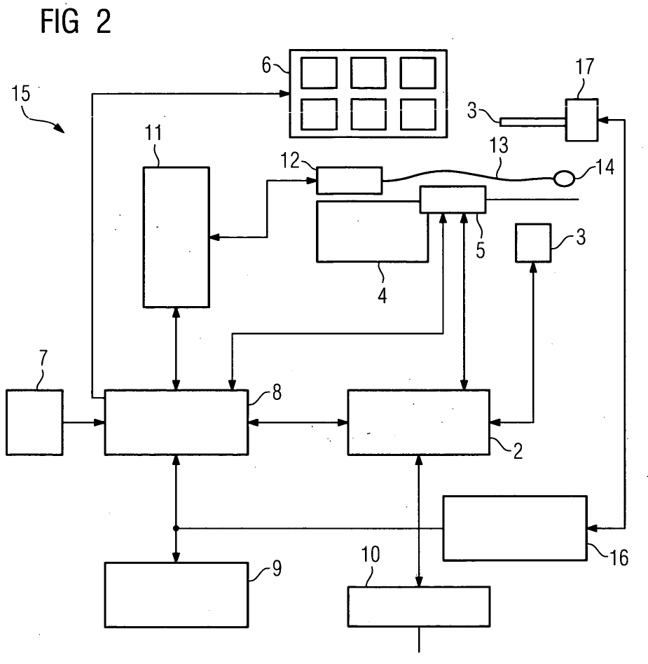

FIG. 2 shows a second embodiment of a medical examination and/or treatment apparatus.

The components also present in the embodiment illustrated in FIG. 1 are denoted by the same reference numerals.

The medical examination and/or treatment apparatus 15 shown in FIG. 2 has essentially the same construction as the apparatus shown in the first embodiment. In addition, a tracking system 16 is provided which is connected to tracking sensors 17 which can detect the position of the magnetic coils 14 and therefore the position of the tip of the catheter 13. The tracking system 16 is linked to the multi-modality interface 8 which is responsible for analyzing this information.

Variants are also conceivable in which the sensor device 11 is incorporated in the multi-modality interface 8, in particular the information supplied by the catheters can be controlled and processed in the multi-modality interface 8. It is also possible for the multi-modality interface to be implemented as a component of the x-ray image recording device 2 or to share resources with the x-ray image recording device 2.

In a typical workflow in a hospital, patient data is first retrieved from a database via the network 10 or manually entered on the x-ray image recording device 2. After the start of the examination a decision is made regarding the use of a specific invasive imaging method. This involves selecting a particular catheter 13. The corresponding plug of this catheter is inserted in the multi-modality interface 8. The patient data is transferred from the x-ray image recording device via the multi-modality interface 8 to the sensor device 11 of the relevant catheter. Then the actual examination takes place, and the examination result can be displayed separately or possibly together with x-ray recordings on a monitor or on the monitor array 6. The physician picks out essential results and causes them to be forwarded to the x-ray image recording device 2 for compiling a report. All or selected results are stored in a database via the network 10 for documentation.

1.-7. (canceled)

8. A medical apparatus for a medical examination on a patient, comprising:
a multi-modality interface that functions as a universal interface;
an x-ray image recording device connected to the medical apparatus via the multi-modality interface;
an image generating device connected to the medical apparatus via the multi-modality interface; and
an invasive sensor device connected to the medical apparatus via the multi-modality interface.

9. The medical apparatus as claimed in claim 8, wherein a plurality of different invasive sensor devices are connected to the medical apparatus via the multi-modality interface.

10. The medical apparatus as claimed in claim 8, wherein the invasive sensor device is a flexible device.

11. The medical apparatus as claimed in claim 10, wherein the flexible device is a catheter.

12. The medical apparatus as claimed in claim 8, wherein the invasive sensor device is a fixed device.

13. The medical apparatus as claimed in claim 12, wherein the fixed device is a needle.

14. The medical apparatus as claimed in claim 8, wherein the multi-modality interface comprises 2 plug in connection.

15. The medical apparatus as claimed in claim 8, wherein the invasive sensor device comprises an image recording system.

16. The medical apparatus as claimed in claim 8, wherein the invasive sensor device is selected from the group consisting of: IVUS, OCT, OFDI, ICE, pressure wire, angioscopy, NIRF imaging, and intravascular MR.

17. The medical apparatus as claimed in claim 8, further comprising a tracking system connected to the multi-modality interface that tracks a position of the invasive sensor device.

18. The medical apparatus as claimed in claim 8, further comprising a network through which a computer is connected to the medical apparatus via the multi-modality interface.

19. The medical apparatus as claimed in claim 8, further comprising an interface selected from the group consisting of: a user interface, an interface to an evaluating computer, an interface to a tracking system, an interface to a magnetic navigation device, an interface to an ECG machine, and combinations thereof.

20. The medical apparatus as claimed in claim 19, wherein the user interface comprises an element selected from the group consisting of: a touch sensitive monitor, a joystick, an operator button, and combinations thereof.

21. The medical apparatus as claimed in claim 8, wherein the medical apparatus is for a medical treatment on the patient.

22. A method for integrating a medical apparatus, comprising:
arrange a multi-modality interface in the medical apparatus as a universal interface;
connecting an x-ray image recording device to the medical apparatus via the multi-modality interface;
connecting an image generating device to the medical apparatus via the multi-modality interface; and
connecting an invasive sensor device to the medical apparatus via the multi-modality interface.
